SEQUENCE LISTING (2 sequences)

SEQ ID NO: 1 - DNA, 411 bp, Cupriavidus necator
  source 1..411: mol_type = other DNA
atgagcattattaaagaatttcgcgaatttgcgatgcgcgggaacgtggtggatttggcggtgggtgtcattatcggtgcggcattcgggaagattgtctcttcactggttgccgatatcatcatgcctcctctgggcttattaattggcgggatcgattttaaacagtttgctgtcacgctacgcgatgcgcagggggatatccctgctgttgtgatgcattacggtgtcttcattcaaaacgtctttgattttctgattgtggcctttgccatctttatggcgattaagctaatcaacaaactgaatcggaaaaaagaagaaccagcagccgcacctgcaccaactaaagaagaagtattactgacagaaattcgtgatttgctgaaagagcagaataaccgctcttaa

SEQ ID NO: 2 - protein, 136 aa, Cupriavidus necator
MSIIKEFREFAMRGNVVDLAVGVIIGAAFGKIVSSLVADIIMPPLGLLIGGIDFKQFAVTLRDAQGDIPAVVMHYGVFIQNVFDFLIVAFAIFMAIKLINKLNRKKEEPAAAPAPTKEEVLLTEIRDLLKEQNNRS

(19) United States
(12) Patent Application Publication
(10) Pub. No.: US 2025/0346909 A1
(43) Pub. Date: Nov. 13, 2025

(54) OSMOLYSIS-BASED RECOVERY OF BIOMACROMOLECULES FROM ENGINEERED HALOTOLERANT MICROORGANISMS

(71) Applicant: The Regents of the University of California, Oakland, CA (US)

(72) Inventors: Douglas S. Clark, Berkeley, CA (US); Jeremy David Adams, Berkeley, CA (US)

(21) Appl. No.: 18/860,177

(22) PCT Filed: Apr. 28, 2023
(86) PCT No.: PCT/US2023/066344

§ 371 Date: Oct. 25, 2024

Related U.S. Application Data
(60) Provisional application No. 63/337,036, filed on Apr. 29, 2022.

(51) Int. Cl. C12N 15/70; C07K 14/245; C12N 15/74; C12R 1/19

(52) U.S. Cl. CPC C12N 15/70; C07K 14/245; C12N 15/74; C12R 2001/19

(57) ABSTRACT

Provided herein are engineered halotolerant microorganisms that are susceptible to cell lysis upon resuspension in distilled water, the process of engineering the halotolerant microorganisms, and the uses thereof, including for the recovery of biomacromolecules.

### CROSS REFERENCE TO RELATED APPLICATIONS

This application claims priority to U.S. Provisional Application Ser. No. 63/337,036, filed Apr. 29, 2022, the disclosures of which are incorporated herein by reference in its entirety.

### STATEMENT OF GOVERNMENT SUPPORT

This invention was made with Government support under Grant No. NNX17AJ31G, awarded by the National Aeronautics and Space Administration. The Government has certain rights in the invention.

### TECHNICAL FIELD

Provided herein are engineered halotolerant microorganisms that are susceptible to cell lysis upon resuspension in distilled water, the process of engineering the halotolerant microorganisms, and the uses thereof, including for the recovery of biomacromolecules.

### INCORPORATION BY REFERENCE OF SEQUENCE LISTING

Accompanying this filing is a Sequence Listing entitled “00012-083WO1.xml”, created on Apr. 28, 2023, and having 3,438 bytes of data, machine formatted on IBM-PC, MS-Windows operating system. The sequence listing is hereby incorporated herein by reference in its entirety for all purposes.

### BACKGROUND

Intracellular biomacromolecules, such as industrial enzymes and biopolymers, represent an important class of bio-derived products. Whether for industrial applications or research purposes, microbial cells containing these molecules must be lysed prior to downstream purification of the bioproduct. Traditionally, on both lab- and industrial-scales, cell lysis is achieved through either physical disruption (e.g., sonication, French press, homogenization) or reagent-based lysis (usually using detergents). Physical disruption methods usually require expensive equipment, have high energy demands, and in some cases may damage the product of interest. Reagent-based methods tend to be milder but can be costly and difficult to scale. Furthermore, the equipment and material requirements significantly raise research costs, which can pose barriers for biomolecular research in labs lacking adequate equipment. Previous work has explored the use of halophilic bacteria such as Halomonas sp. to produce intracellular biomolecules, specifically biopolymers, due to the susceptibility of such strains to lyse when resuspended in distilled water owing to the osmotic pressure drops. However, the majority of industrial microbial strains are non-halophilic, limiting the utility of this strategy.

### SUMMARY

Intracellular biomacromolecules represent an important class of bio-derived commodity products, and include products such as industrial enzymes and biopolymers. Whether purified for industrial or research purposes, cells containing these molecules must be lysed prior to downstream purification of the desired end-product. This invention simplifies this process by developing bacterial strains that are susceptible to lysis in distilled water. Two orthogonal strategies enable this strain development. First, the strain is adapted to grow in higher salt concentrations by adaptive laboratory evolution, enabling greater osmotic pressure upon resuspension in distilled water. Second, the gene encoding the large conductance mechanosensitive channel (mscL) is knocked out, mitigating the native osmotic shock survival mechanism. The combination of these two strategies causes significant cell lysis to occur in distilled water and enables the release and recovery of the desired biomolecule product.

The disclosure provides an engineered microorganism that is halotolerant or adapted to become halotolerant and comprises a knockout of a Large- and/or Small-conductance mechanosensitive channel gene. In one embodiment, the microorganism can grow in greater than 1.5% w/v NaCl to about 4.0% w/v NaCl. In still another or further embodiment, the engineered microorganism can grow in greater than 2.0% w/v NaCl to about 4.0% w/v NaCl. In still a further embodiment of any of the foregoing embodiments, the engineered microorganism can grow in greater than 3.0% w/v NaCl to about 4.0% w/v NaCl. In still further embodiments, the large-conductance mechanosensitive channel gene is mscL gene or a homolog thereof. In still a further embodiment, the small-conductance mechanosensitive channel gene is mscS. In still further embodiments, the mscL gene has a sequence of SEQ ID NO:1 or a sequence that is at least 80% identical to SEQ ID NO:1 and has mechanosensitive channel activity similar to wild-type mscL activity. In still another embodiment, the large-conductance mechanosensitive channel gene encodes a polypeptide that is at least 85% identical to SEQ ID NO:2. In still further embodiments, the microorganism is adapted to grow on a salt medium of 1.5% to about 3.25% (w/v) NaCl prior to knocking out the Large- and/or small-conductance mechanosensitive channel gene. In still another embodiment, the engineered microorganism is further engineered to produce a non-natural chemical or bioproduct.

The disclosure also provide a method of producing a desired recombinant protein comprising transforming an engineered halotolerant microorganism of the disclosure with a vector encoding the desired recombinant protein. The disclosure also provides a method of producing a desired chemical compound comprising transforming an engineered microorganism of the disclosure with one or more polynucleotide(s) encoding a polypeptide or polypeptides that provides for the synthesis of the desired chemical compound. In a further embodiment of either of the foregoing embodiments, the engineered microorganism is cultured to produce the desired recombinant protein or the desired chemical compound. In a further embodiment, the method can further comprise passaging/transferring the microorganism to a hypotonic solution to lyse the microorganism; and isolating the desired recombinant protein or the desired chemical compound released from the engineered microorganism.

The disclosure also provides a method of generating a halotolerant microorganism from a non-halotolerant microorganism, the method comprising: (a) passaging the non-halotolerant microorganisms on media that increases in salt concentration from by 0.25% (w/v) NaCl until reaching a final media concentration of about 1.5% to 3.0% (w/v) NaCl to obtain a laboratory evolved halotolerant strain; (b) engineering the laboratory evolved halotolerant strain by genetically knocking out a gene(s) encoding a large and/or small conductance mechanosensitive channel protein(s) or a homolog thereof to obtain a halotolerant microorganism. In one embodiment, the non-halotolerant microorganism is C. necator. In another embodiment, the non-halotolerant microorganism is E. coli. In still another or further embodiment, the large conductance mechanosensitive channel gene is mscL. In still another or further embodiment, the small conductance mechanosensitive channel gene is mscS. In still a further embodiment, the microorganism has greater than 75-90% osmolytic efficiency upon osmotic downshock.

The disclosure also provides a halotolerant C. necator strain that grows on (i) 3% NaCl luria burtani broth (LB) or (ii) M9 formate with 16 g/L NaCl. In a further embodiment, the strain lacks expression of a large conductance mechanosensitive channel protein.

The disclosure also provides a halotolerant E. coli strain comprising ΔmscL and ΔmscS.

In a particular embodiment, the disclosure provides an engineered halotolerant microorganism comprising a knockout of a Large-conductance mechanosensitive channel gene. In a further embodiment, the engineered halotolerant microorganism can grow with greater than 1.5% w/v NaCl. In a further embodiment, the engineered halotolerant microorganism can grow with greater than 2.0% w/v NaCl. In yet a further embodiment, the engineered halotolerant microorganism can grow with greater than 3% w/v NaCl. In another embodiment, the large-conductance mechanosensitive channel gene is mscL gene or a homolog thereof. In yet another embodiment, the mscL gene has a sequence of (SEQ ID NO:1):

| | |
|---|---|
| 1 | atgagcatta ttaaagaatt tcgcgaattt gcgatgcgcg ggaacgtggt ggatttggcg |
| 61 | gtgggtgtca ttatcggtgc ggcattcggg aagattgtct cttcactggt tgccgatatc |
| 121 | atcatgcctc ctctgggctt attaattggc gggatcgatt ttaaacagtt tgctgtcacg |
| 181 | ctacgcgatg cgcaggggga tatccctgct gttgtgatgc attacggtgt cttcattcaa |
| 241 | aacgtctttg attttctgat tgtggccttt gccatcttta tggcgattaa gctaatcaac |
| 301 | aaactgaatc ggaaaaaaga agaaccagca gccgcacctg caccaactaa agaagaagta |
| 361 | ttactgacag aaattcgtga tttgctgaaa gagcagaata accgctctta a |

In a certain embodiment, the mscL gene homolog is at least 80% identical to (SEQ ID NO:1):

| | |
|---|---|
| 1 | atgagcatta ttaaagaatt tcgcgaattt gcgatgcgcg ggaacgtggt ggatttggcg |
| 61 | gtgggtgtca ttatcggtgc ggcattcggg aagattgtct cttcactggt tgccgatatc |
| 121 | atcatgcctc ctctgggctt attaattggc gggatcgatt ttaaacagtt tgctgtcacg |
| 181 | ctacgcgatg cgcaggggga tatccctgct gttgtgatgc attacggtgt cttcattcaa |
| 241 | aacgtctttg attttctgat tgtggccttt gccatcttta tggcgattaa gctaatcaac |
| 301 | aaactgaatc ggaaaaaaga agaaccagca gccgcacctg caccaactaa agaagaagta |
| 361 | ttactgacag aaattcgtga tttgctgaaa gagcagaata accgctctta a |

In a further embodiment, the large-conductance mechanosensitive channel gene encodes a polypeptide that is at least 85% identical to:

(SEQ ID NO: 2)
MSIIKEFREFAMRGNVVDLAVGVIIGAAFGKIVSSLVADIIMPPLG
LLIGGIDFKQFAVTLRDAQGDIPAVVMHYGVFIQNVFDFLIVAFAI
FMAIKLINKLNRKKEEPAAAPAPTKEEVLLTEIRDLLKEQNNRS.

In a certain embodiment, the disclosure also provides a process or method of producing a desired recombinant protein comprising transforming an engineered osmotically susceptible halotolerant microorganism disclosed herein with a vector encoding the desired recombinant protein. In a particular embodiment, the disclosure further provides a process or method of producing a desired chemical compound comprising transforming an engineered halotolerant microorganism disclosed herein with one or more polynucleotide(s) encoding a polypeptide or polypeptides that provides for the synthesis of the desired chemical compound. In another embodiment engineered halotolerant microorganism is cultured to produce the desired recombinant protein or the desired chemical compound. In yet another embodiment, the method further comprises: transferring the engineered halotolerant microorganism to a hypotonic solution to lyse the microorganism; and isolating the desired recombinant protein or the desired chemical compound released from the engineered halotolerant microorganism.

In a particular embodiment, the disclosure provides for an engineered halotolerant microorganism substantially described and/or shown herein.

In a certain embodiment, the disclosure provides for a process or method to make an engineered halotolerant microorganism substantially described and/or shown herein.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-B shows an Overview of approach. (A) Schematic representation of osmolysis-based recovery of intracellular biomacromolecules. Product is first produced by microbial host under elevated salt concentrations. Cells are then resuspended in distilled water, causing an increase of turgor pressure due to osmotic shock, which lyses the cell membrane and enables downstream recovery of the product. (B) Two orthogonal strategies employed here to increase the sensitivity of microbial hosts to osmotic downshock. In one strategy (i) ALE is used to increase the halotolerance of the microbe, enabling cell growth at higher salt concentrations and therefore a greater magnitude of osmotic downshock when cells are resuspended in distilled water. In the other strategy (ii) the large-conductance mechanosensitive channel (mscL) or a related gene is deleted from the microbial host, which limits the ability of cells to export osmolytes in hypotonic solutions, increasing their susceptibility to osmotic lysis.

FIG. 2A-B shows adaptive laboratory evolution yields halotolerant strain of C. necator. (A) Results of ALE experiment showing the growth rate (blue bars) and NaCl concentration (red line) at each passage. (B) Growth curve of wild-type C. necator strain H16 and evolved strain ht030b in LB containing 3% NaCl (w/v, final concentration). For each data point in (B), mean±SD is displayed (n=4).

FIG. 3A-B shows osmolysis efficiency, calculated as a fraction of RFP recovered in supernatant compared to total RFP content following osmotic shock (see methods), as a function of osmotic downshock magnitude for wild-type C. necator H16 (circles) and mutant C. necator H16 ΔmscL (diamonds) following growth in (A) LB with 1.5% (w/v) NaCl (final concentration) and (B) M9 sodium formate (4 g/L) medium supplemented with 6 g/L NaCl. Differences in values on the x-axis of the two graphs reflect slight differences in starting osmolarities of the two media tested. For each data point, mean±SD is displayed (n=6).

FIG. 4A-B shows the effect of combined mscL gene knockout and improved halotolerance on osmolysis in C. necator. Osmolysis efficiency, calculated as a fraction of RFP recovered in cell-free supernatant compared to total RFP content following osmotic shock (relative concentrations determined by fluorescence intensity), as a function of osmotic downshock magnitude for ht030b (circles) and ht030b ΔmscL (diamonds) following growth in (A) LB with 3.0% NaCl (w/v, final concentration) and (B) M9 sodium formate (4 g/L) medium supplemented with 16 g/L NaCl. Differences in values on the x-axis of the two graphs reflect differences in starting osmolarities of the two media tested. For each data point, mean±SD is displayed (n=6).

FIG. 5A-C shows the application of mechanosensitive knockout for osmolysis in E. coli BL21. (A) Percent cell lysis following growth in LB with 4% NaCl of BL21 (left bar of each set), BL21 ΔmscL (middle bar of each set), and BL21 ΔmscL ΔmscS (right bar of each set) in three different media: commercial B-PER™ Bacterial Protein Extraction Reagent; a 4% NaCl(aq) isotonic solution; and distilled water (diH2O). Cell lysis is determined as the fraction of RFP recovered in supernatant compared to total RFP content, as it was in C. necator, with concentration measured by fluorescence intensity (n=6). (B) Growth rate (diamonds) and osmolysis efficiency (circles) of BL21 ΔmscL ΔmscS in LB with various (final) NaCl concentrations (n=3). (C) SDS-PAGE gel of various fractions of RFP-expressing BL21 and BL21 ΔmscL ΔmscS. NE: cells not expressing RFP, WC: whole-cell fraction, S: post-osmolysis supernatant fraction, P: post-osmolysis cell pellet. For each data point in (A) and (B), mean±SD is displayed.

FIG. 6 shows growth curve of H16 (circles) and ht030b (diamonds) in LB containing 3.25% NaCl in 50-mL cultures in shake flasks, seeded at an optical density of A₆₀₀ nm=0.05.

FIG. 7 shows growth curve of C. necator H16 (circles) and H16 ΔmscL (diamonds).

FIG. 8A-D shows optical densities of C. necator H16 (A) and C. necator ht030b (B) following 24 hours of growth in LB at various salt (final) concentrations as well as H16 (C) and ht030b (D) following 48 hours of growth in M9 formate with various (added) salt concentrations. Black dashed line represents cutoff OD of 0.22 (LB growth) and 0.077 (M9 growth) which defines thresholds of salt tolerance in the respective media.

FIG. 9A-C shows (A) Schematic overview of RFP assay as described. Well-mixed red fluorescence measurements (585 nm excitation/620 nm emission) were performed on the well-mixed sample, representing the total RFP content, and from the supernatant following centrifugation, representing the released RFP content. Cell lysis fraction was taken to be the ratio of released RFP to total RFP. (B) Representative linear range validation that was replicated in each experiment to verify that RFP concentration was proportional to fluorescence intensity. (C) Fluorescence intensity measurements of identical RFP-expressing cell samples in various solutions, demonstrating that the fluorescence intensity is not sensitive to the various environments encountered in the assay.

FIG. 10A-B shows (A) Growth of E. coli BL21 at various salt concentrations as a function of time. (B) Semilog of cell density as a function of time during logarithmic growth phase.

FIG. 11 shows the effect of addition of freeze-thaw step (right bar) with osmolysis for BL21 and BL21 ΔmscL ΔmscS compared to cells only subjected to osmotic downshock (left bar).

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the enzyme” includes reference to one or more enzymes, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Any publications discussed above and throughout the text are provided solely for their disclosure prior to the filing date of the present application. Nothing herein is to be construed as an admission that the inventors are not entitled to antedate such disclosure by virtue of prior disclosure.

Whole-cell biocatalysis encompasses a wide range of existing processes in which microbes convert a feedstock to a desirable product (e.g., a low-value feedstocks to higher-value products). Biochemical processes can produce biomolecules such as proteins that cannot be produced by traditional chemical processes, as well as fuels, commodity chemicals, and bioplastics that would otherwise be produced in petrochemical processes that contribute to climate change. Downstream separations of the desired product are an important and often costly component of any bioprocess and can vary significantly depending on whether the product of interest is extracellular or intracellular. Certain industrial microbial hosts, for example Bacillus subtilis and Bacillus licheniformis, secrete enzymes such as proteases with high yields to the extracellular space, which allows for relatively simple purification of these biomolecules. However, such strategies are limited to specific proteins produced in certain strains, as not all biomolecular products are suited for transport across the cell membrane. Intracellular macromolecular bioproducts on the other hand can be challenging to separate from bacterial biomass as these molecules cannot easily diffuse through the cell membrane, and therefore require cellular disruption to recover the product.

Intracellular macromolecules represent an important class of bioproducts. For example, recombinant proteins (industrial enzymes and biopharmaceuticals, in particular) are widely used intracellular products. The demand for high-quality plasmid DNA, generally produced as an intracellular product in bacteria such as E. coli, has greatly increased as more cell and gene therapies have been developed. Certain bioplastics such as polyhydroxyalkanoates (PHAs) are produced as full-length polymers in many bacteria. PHAs, most notably polyhydroxybutyrate (PHB), are native products in many bacteria where the bacterial use it as a store of carbon and energy. Non-native PHB producers, such as E. coli, have also been engineered for PHB production.

Traditional bio-separations of biomacromolecules use cell lysis prior to downstream purification of the desired product. Mechanical methods such as ultrasonication and high-pressure homogenization can efficiently lyse cells, though these require expensive equipment, are energy-intensive, and may damage sensitive biomolecules. Chemical and enzymatic methods of cell lysis can also be used to liberate intracellular products, though the cost of the materials make these techniques difficult to scale.

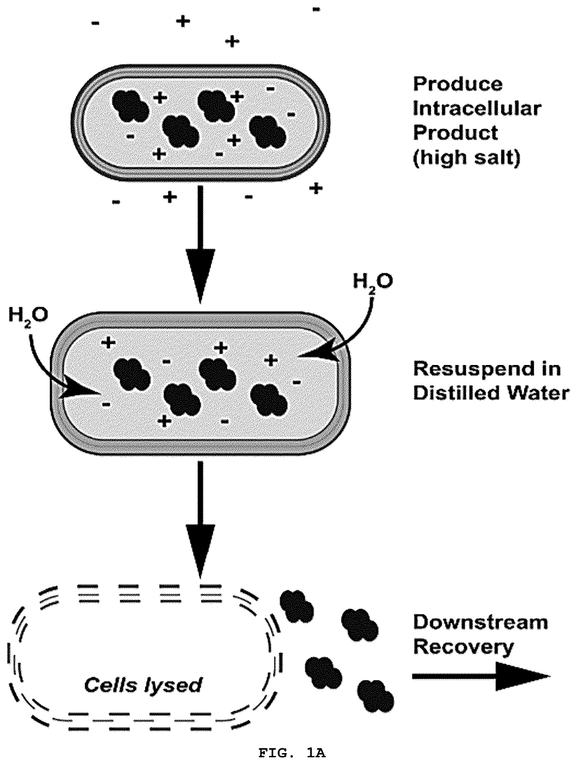

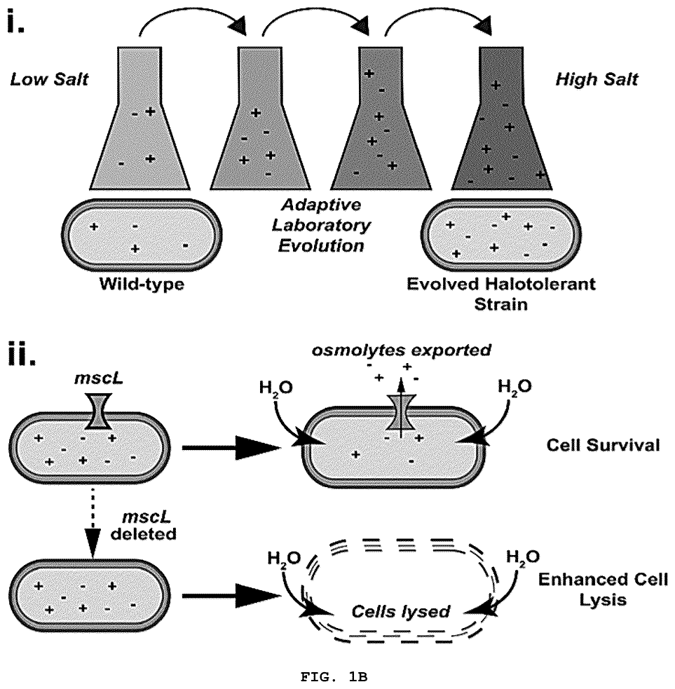

Osmolysis is a simple, low-cost method of cell lysis that relies on osmotic pressure to swell cells and burst membranes following the resuspension of cells in a hypotonic solution (FIG. 1A). Osmolysis as a cell lysis technique in downstream separations has traditionally been restricted to mammalian cell culture, where the weaker cell membrane is fairly labile to osmotic pressure changes. The more robust bacterial cell wall, as well as stress-response survival mechanisms in bacteria, allow most bacteria to survive moderate fluctuations of osmolarity. Extreme halophiles can grow in salinities from 15 to 30% NaCl (w/v), and therefore resuspension of these microbes in distilled water will cause a much higher osmotic pressure shock than can be achieved with bacteria grown in conventional media. However, extremely halophilic bacteria are rarely, if at all, used in industrial bioprocesses, and many applications call for specific bacterial strains that are likely not halophilic.

Electromicrobial production (EMP) is an emerging technology with the potential to generate a wide array of useful bioproducts. EMP relies on bacteria that utilize electricity or electrochemically generated mediator molecules such as hydrogen gas and formic acid as energy sources to produce various bioproducts. Traditional biochemical systems use crop-derived sugars as microbial substrates and therefore cause social and environmental impacts such as carbon emissions from fertilizer production, nitrous oxide emissions from fertilizer application, land use effects, and competition with the food supply. EMP systems, however, do not rely on the agricultural system, and, if using a clean electricity source, can lead to a decreased global warming potential and land occupation footprint.

One microbe studied for EMP systems is Cupriavidus necator, a soil bacterium capable of growth on various substrates, including H₂/CO₂, formate, and organic molecules. Electrolysis of water to produce hydrogen or electrochemical reduction of carbon dioxide to formic acid can therefore be used to generate substrates that Knallgas or formatotrophic bacteria (both of which describe C. necator) can convert to desired products. C. necator naturally produces the polyester PHB, and is often regarded as a model organism for PHA production due to its ability to accumulate high levels of the polymer intracellularly (up to 90% of total cell mass) and its potential in producing many PHA variants. In addition, expression systems have been developed for C. necator that allow production of recombinant proteins, and metabolic engineering has been applied to produce various fuels and commodity chemicals such as isopropanol, acetoin, and various alkanes.

While EMP addresses the environmental impacts of substrate generation in bioprocessing, a sustainable bioproduction system must also minimize energy and resource demand during separations. Adapting the osmolysis cell disruption method to work with EMP-relevant microbes could address the resource-intensive separations process for intracellular macromolecular products produced through EMP. However, no EMP systems have used halophilic or halotolerant bacteria.

The disclosure provides a two-part strategy to render non-halophilic bacteria susceptible to lysis by osmotic downshock, using C. necator, a microbe commonly used in EMP systems, as an example host microbe (FIG. 1B). The disclosure demonstrates that intracellular biomolecule products can be separated from the cells, using recombinant red fluorescent protein (RFP) as a useful example product due to its ease of measurement.

As an example, the disclosure used adaptive laboratory evolution (ALE) to improve the halotolerance of C. necator, which enables a greater magnitude of osmolarity change and therefore greater osmotic pressure when the cells are resuspended in distilled water. In parallel, C. necator was engineered by knocking out the large-conductance mechanosensitive channel (mscL) gene, a membrane protein that facilitates cell survival during hypotonic shock that is found in a wide range of bacteria. While either method individually can improve the susceptibility of the bacteria to osmolysis, the disclosure demonstrates that combining these two methods in a single strain (e.g., one that is both halotolerant and lacks the mscL gene or homolog thereof) enables significantly higher osmolytic efficiency than either method individually. The osmolytic efficiency of cells lysed upon osmotic downshock was assayed using an RFP-based assay to determine the fraction of intracellular contents released to the media. In addition, the disclosure demonstrates that the methods and resulting engineered bacteria can be expanded to other bacteria by adapting the methods of the disclosure to E. coli BL21, a strain routinely used in the production of recombinant proteins. Both the mscL gene, and the related small-conductance mechanosensitive channel (mscS) gene are knocked out of BL21 to make it susceptible to osmolysis.

As used herein, an “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product. The disclosure provides recombinant microorganism having a metabolically engineered pathway for the production of a desired product or intermediate.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

An “enzyme” means any substance, typically composed wholly or largely of amino acids making up a protein or polypeptide that catalyzes or promotes, more or less specifically, one or more chemical or biochemical reactions.

The term “expression” with respect to a gene or polynucleotide refers to transcription of the gene or polynucleotide and, as appropriate, translation of the resulting mRNA transcript to a protein or polypeptide. Thus, as will be clear from the context, expression of a protein or polypeptide results from transcription and translation of the open reading frame.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

In addition, and as mentioned above, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

As used herein, two proteins (or a region of the proteins) are substantially homologous when the amino acid sequences share at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

A typical algorithm used for comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than BLASTp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

In some instances “isozymes” can be used that carry out the same functional conversion/reaction, but which are so dissimilar in structure that they are typically determined to not be “homologous”.

A “mechanosensitive channel” is a pore forming membrane protein that can modulate salt flux. Mechanosensitive channels as used herein include the large conductance mechanosensitive channels (mscL) and the small conductance mechanosensitive channels (mscS). Similar to other ion channels, MscLs are organized as symmetric oligomers with the permeation pathway formed by the packing of subunits around the axis of rotational symmetry. Unlike MscS, which is heptameric, MscL are typically pentameric. MscL contains two transmembrane helices that are packed in an up-down/nearest neighbor topology. The permeation pathway of the MscL is approximately funnel shaped, with larger opening facing the periplasmic surface of the membrane and the narrowest point near the cytoplasm. At the narrowest point, the pore is constricted by the side chains of symmetry-related residues in E. coli-MscL: Leu19 and Val23. The E. coli mscL consists of five identical subunits, each 136 amino acids long. Each subunit crosses the membrane twice through alpha-helical transmembrane segments, which are interconnected by an extracellular loop.

As used herein, the term “metabolically engineered” or “metabolic engineering” involves rational pathway design and assembly of biosynthetic genes, genes associated with operons, and control elements of such polynucleotides, for the production of a desired metabolite in a microorganism, partially in a microorganism, in a cell free system and/or a combination of cell-free system and microorganism. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

A “metabolite” refers to any substance produced by metabolism or enzymatic pathway or a substance necessary for or taking part in a particular metabolic process or pathway that gives rise to a desired metabolite, chemical, etc. A metabolite can be an organic compound that is a starting material (e.g., glucose etc.), an intermediate in (e.g., glyceraldehyde-3-phosphate), or an end product (e.g., pyruvate) of metabolism or enzymatic pathway. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A “mutation” means any process or mechanism by which a protein, enzyme, polynucleotide, gene or cell is modified to change a parental protein, enzyme, polynucleotide, gene or cell. This includes any mutation in which a protein, enzyme, polynucleotide, or gene sequence is altered, and any detectable change in a cell arising from such a mutation. Typically, a mutation occurs in a polynucleotide or gene sequence, by point mutations, deletions, or insertions of single or multiple nucleotide residues. A mutation includes polynucleotide alterations arising within a protein-encoding region of a gene as well as alterations in regions outside of a protein-encoding sequence, such as, but not limited to, regulatory or promoter sequences. A mutation in a gene can be “silent”, i.e., not reflected in an amino acid alteration upon expression, leading to a “sequence-conservative” variant of the gene. This generally arises when one amino acid corresponds to more than one codon. A mutation that gives rise to a different primary sequence of a protein can be referred to as a mutant protein or protein variant.

A “native” or “wild-type” protein, enzyme, polynucleotide, gene, or cell, means a protein, enzyme, polynucleotide, gene, or cell that occurs in nature.

A “parental microorganism” refers to a cell used to generate a recombinant microorganism. The term “parental microorganism” describes, in one embodiment, a cell that occurs in nature, i.e. a “wild-type” cell that has not been genetically modified. The term “parental microorganism” further describes a cell that serves as the “parent” for further engineering. In this latter embodiment, the cell may have been genetically engineered, but serves as a source for further genetic engineering.

For example, a wild-type microorganism can be genetically modified to express or over express a first target enzyme. This modified microorganism can then act as a parental microorganism in the generation of a microorganism modified to express or over-express a second target enzyme. In turn, that microorganism can be modified to express or over express a third target enzyme, etc. As used herein, “express” or “over express” refers to the phenotypic expression of a desired gene product. In one embodiment, a naturally occurring gene in the organism can be engineered such that it is linked to a heterologous promoter or regulatory domain, wherein the regulatory domain causes expression of the gene, thereby modifying its normal expression relative to the wild-type organism. Alternatively, the organism can be engineered to remove or reduce a repressor function on the gene, thereby modifying its expression. In yet another embodiment, a cassette comprising the gene sequence operably linked to a desired expression control/regulatory element is engineered in to the microorganism. In still further embodiments, the organism can be used to knockout of reduce a first, second or third gene or a combination of knocking-in and knocking out gene expression.

Accordingly, a parental microorganism functions as a reference cell for successive genetic modification events. Each modification event can be accomplished by introducing one or more nucleic acid molecules into the reference cell. The introduction facilitates the expression or over-expression of one or more target enzyme or the reduction or elimination of one or more target enzymes. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme into a parental microorganism.

A “parental enzyme or protein” refers to an enzyme or protein used to generate a variant or mutant enzyme or protein. The term “parental enzyme” (or protein) describes, in one embodiment, an enzyme or protein that occurs in nature, i.e. a “wild-type” enzyme or protein that has not been genetically modified. The term “parental enzyme” (or protein) further describes a cell that serves as the “parent” for further engineering. In this latter embodiment, the enzyme or protein may have been genetically engineered, but serves as a source for further genetic engineering.

The term “polynucleotide,” “nucleic acid” or “recombinant nucleic acid” refers to polynucleotides such as deoxyribonucleic acid (DNA), and, where appropriate, ribonucleic acid (RNA).

Polynucleotides that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. The sequences provided herein and the accession numbers provide those of skill in the art the ability to obtain and obtain coding sequences for various enzymes of the disclosure using readily available software and basic biology knowledge.

Those of skill in the art will recognize that, due to the degenerate nature of the genetic code, a variety of codons differing in their nucleotide sequences can be used to encode a given amino acid. A particular polynucleotide or gene sequence encoding a biosynthetic enzyme or polypeptide described above are referenced herein merely to illustrate an embodiment of the disclosure, and the disclosure includes polynucleotides of any sequence that encode a polypeptide comprising the same amino acid sequence of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate exemplary embodiments of the disclosure.

The disclosure provides polynucleotides in the form of recombinant DNA expression vectors or plasmids, as described in more detail elsewhere herein, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides polynucleotides in isolated (i.e., not pure, but existing in a preparation in an abundance and/or concentration not found in nature) and purified (i.e., substantially free of contaminating materials or substantially free of materials with which the corresponding DNA would be found in nature) form.

A polynucleotide of the disclosure can be amplified using cDNA, mRNA or alternatively, genomic DNA, as a template and appropriate oligonucleotide primers according to standard PCR amplification techniques. The nucleic acid so amplified can be cloned into an appropriate vector and characterized by sequence analysis. Furthermore, oligonucleotides corresponding to nucleotide sequences can be prepared by standard synthetic techniques, e.g., using an automated DNA synthesizer.

The disclosure provides a number of polypeptide sequences in the sequence listing accompanying the present application, which can be used to design, synthesize and/or isolate polynucleotide sequences using the degeneracy of the genetic code or using publicly available databases to search for the coding sequences.

It is also understood that an isolated polynucleotide molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitution, in some positions it is preferable to make conservative amino acid substitutions.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

It is understood that a polynucleotide described herein include “genes” and that the nucleic acid molecules described above include “vectors” or “plasmids.”

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

A “protein” or “polypeptide”, which terms are used interchangeably herein, comprises one or more chains of chemical building blocks called amino acids that are linked together by chemical bonds called peptide bonds. A protein or polypeptide can function as an enzyme.

“Recombinant” refers to polynucleotides manipulated in vitro (“recombinant polynucleotides”) and to methods of using recombinant polynucleotides to produce or inhibit gene products encoded by those in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well. Similarly, a cell can be engineered to have reduced expression of a normal endogenous gene and thus is also considered “recombinant”.

“Transformation” refers to the process by which a vector is introduced into a host cell. Transformation (or transduction, or transfection), can be achieved by any one of a number of means including electroporation, microinjection, biolistics (or particle bombardment-mediated delivery), or Agrobacterium mediated transformation.

A “vector” generally refers to a polynucleotide that can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an Agrobacterium or a bacterium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433, which is incorporated herein by reference in its entirety), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of a gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

The disclosure provides methods or processes for engineering non-halotolerant bacterial strains to make them susceptible to cell lysis upon resuspension in distilled water (osmolysis). The process for osmolysis-based recovery of biomacromolecules is shown in FIG. 1A.

Two orthogonal strategies enable osmolysis susceptibility (e.g., see FIG. 1B). In the first strategy, the microbial strain of interest is adapted to grow in higher salinities through adaptive laboratory evolution by successively growing the microbial strain at increasing salt concentrations (FIG. 1B(i)). In a proof-of-principle study, Cupriavidus necator, a gram-negative soil bacterium was used as a model organism for the production of polyhydroxyalkanoates (PHAs). Using the engineering first strategy, C. necator was evolved to grow in higher salt concentrations. The salt tolerance of C. necator strain H16 was improved from 1.5% to 3.25% w/v NaCl after 30 serial passages. It was then shown that this evolved strain of C. necator was more susceptible to osmolysis than the wild-type strain, due to the higher osmotic pressure drop achievable by resuspending the cells in distilled water.

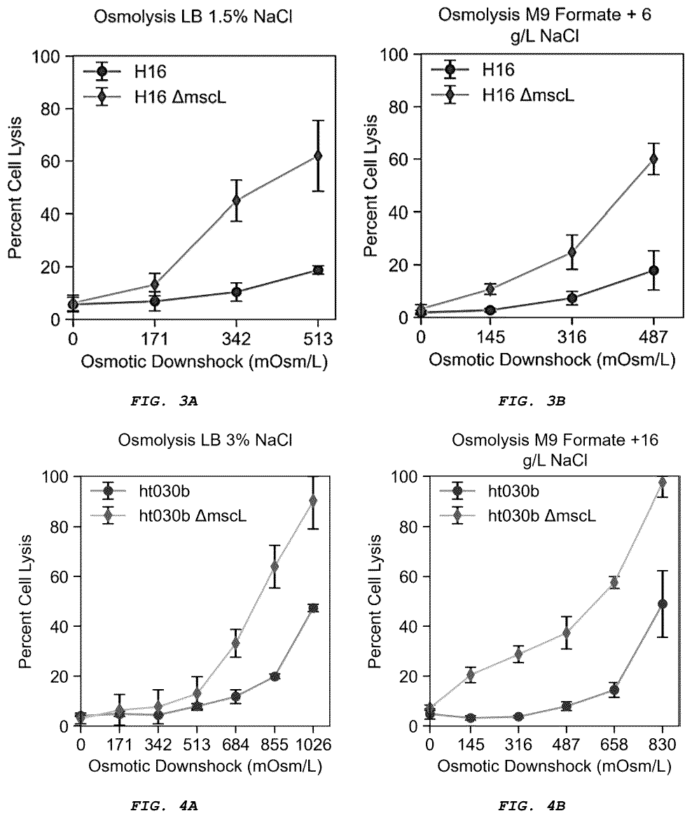

A second strategy (FIG. 1B(ii)) was employed which rendered the microorganisms inherently more susceptible to lysis in distilled water by knocking out the large-conductance mechanosensitive channel (mscL) gene and/or related homologs, which, in many non-halotolerant bacteria, rescues cells from osmotic pressure drops. As demonstrated, C. necator was significantly more susceptible to cell lysis in distilled water when the mscL gene was knocked out (e.g., see FIG. 3A).

To achieve even higher degrees of cell lysis, the two major strategies employed to enable osmolysis were then combined in a single strain by knocking out the mscL gene in an evolved halotolerant strain, C. necator ht030b. As shown in FIG. 4A, the combination of these two approaches in C. necator yielded an osmolysis efficiency of >99%, enabling nearly complete release of the desired biomolecule from the bacterial cells following resuspension in distilled water.

Accordingly, the disclosure provides processes that can be used to develop halotolerant microbial strains that are susceptible to lysis in distilled water. Two orthogonal strategies can be used alone or in combination for microbial strain development. First, the microbial strain is adapted to grow in higher salt concentrations by adaptive laboratory evolution, enabling greater osmotic pressure upon resuspension in distilled water. Second, the gene encoding the large conductance mechanosensitive channel (mscL) and/or homologs thereof (e.g., mscS) is knocked out, mitigating the native osmotic shock survival mechanism. The combination of these two strategies causes significant cell lysis to occur in distilled water and enables the release and recovery of the desired biomolecule product from the engineered halotolerant microorganism.

In a particular embodiment, the disclosure provides for an engineered halotolerant microorganism comprising a knockout of a large-conductance mechanosensitive channel gene or homologs thereof. The process of engineering a microorganism to be halotolerant can include the processes and methods disclosed herein. For example, the microorganism may undergo an adaptive laboratory evolution selection process to select for strains of microorganisms that have are able to grow and proliferate in successively higher salinity concentrations. As shown in the examples presented herein, an engineered strain of Cupriavidus necator was identified and selected that was able to grow and proliferate at a much higher salinity concentration than the wild type Cupriavidus necator. Using such a laboratory evolution selection process can be applied generally to all types of microorganisms and is not specifically limited to Cupriavidus necator. For example, many different types of microorganisms, such as bacteria, archaea, fungi, etc. can use the methods and processes disclosed herein to identify halotolerant strains. Particular examples of microorganisms that can be used with the processes and methods disclosed herein include, Cupriavidus necator, Escherichia coli, Saccharomyces cerevisiae, Aspergillus niger, Clostridium butyclicum, Clostridium thermocellus, Thermus aquaticus, Bacillus coagulans, Bacillus thuringiensis, Xanthomonas campestri, Deinococcus radiorans, Pseudomonas stutzeri, and Leuconostoc mesenteroides.

In a particular embodiment, the engineered halotolerant microorganism disclosed herein is a bacterium. In a further embodiment, the engineered halotolerant microorganism disclosed herein is a gram-negative bacterium. By using the process or methods disclosed herein, a microorganism can be adapted to grow on, with or in a NaCl of/or greater than 1.0% w/v, 1.2% w/v, 1.4% w/v, 1.5% w/v, 1.6% w/v, 1.7% w/v, 1.8% w/v, 2.0% w/v, 2.2% w/v, 2.4% w/v, 2.5% w/v, 2.6% w/v, 2.8% w/v, 3.0% w/v, 3.2% w/v, 3.4% w/v, 3.5% w/v, 3.6% w/v, 3.8% w/v, 4.0% w/v, 4.2% w/v, 4.4% w/v, 4.5% w/v, 4.6% w/v, 4.8% w/v, or in a range that is between or includes any two of the foregoing percentages.

In a further embodiment, the disclosure provides for an engineered halotolerant microorganism comprising a knockout of a Large-conductance mechanosensitive channel gene or homologs thereof. The large-conductance mechanosensitive channel gene where the large-conductance mechanosensitive channel gene is mscL gene or a homolog thereof. The mscL gene is found in many types of bacteria, such as Cupriavidus necator, E. coli, Staphylococcus aureus, Mycobacterium tuberculosis, etc. In a particular embodiment, the mscL gene or mscL gene homolog has a sequence that is at least 80%, 85%, 90%, 95%, 98%, 99% or 100% identical to the sequence of (SEQ ID NO:1):

| | |
|---|---|
| 1 | atgagcatta ttaaagaatt tcgcgaattt gcgatgcgcg ggaacgtggt ggatttggcg |
| 61 | gtgggtgtca ttatcggtgc ggcattcggg aagattgtct cttcactggt tgccgatatc |
| 121 | atcatgcctc ctctgggctt attaattggc gggatcgatt ttaaacagtt tgctgtcacg |
| 181 | ctacgcgatg cgcaggggga tatccctgct gttgtgatgc attacggtgt cttcattcaa |
| 241 | aacgtctttg attttctgat tgtggccttt gccatcttta tggcgattaa gctaatcaac |
| 301 | aaactgaatc ggaaaaaaga agaaccagca gccgcacctg caccaactaa agaagaagta |
| 361 | ttactgacag aaattcgtga tttgctgaaa gagcagaata accgctctta a. |

In a further embodiment, the large-conductance mechanosensitive channel gene encodes a polypeptide that is at least 80%, 85%, 90%, 95%, 98%, 99% or 100% identical to (SEQ ID NO:2):

MSIIKEFREFAMRGNVVDLAVGVIIGAAFGKIVSSLVADIIMPPLG
LLIGGIDFKQFAVTLRDAQGDIPAVVMHYGVFIQNVFDFLIVAFAI
FMAIKLINKLNRKKEEPAAAPAPTKEEVLLTEIRDLLKEQNNRS.

The following table provides examples of microorganisms (order or species) that include large conductance mechanosensitive channels having at least 98% identity to SEQ ID NO:2. The organisms set forth in the table below can also be engineered and adapted as described herein.

| Organism | % identity to SEQ ID NO:2 | Accession number |
|---|---|---|
| Enterobacterales | 100% | WP000022442.1 |
| E. coli | 99.26% | WP179806072.1 |
| E. fergusonii | 99.26% | EHJ4142058.1 |
| E. albertii | 98.53% | EEW7497959.1 |
| Citrobacter | 98.53% | WP042326268.1 |
| Shigella sp. | 99.26% | HAK08127.1 |
| E. marmotae | 98.53% | WP181478720.1 |
| Salmonella enterica | | MIQ94245 |

(See also Blount et al., Microbiol. Mol. Biol. Rev., 84(1):e00055-19, 2020, for a general description of mechanosensitive channels and homologs, the disclosure of which is incorporated herein by reference).

The also disclosure provides methods or processes for producing an engineered halotolerant microorganism disclosed herein that expresses a desired protein or compound. A process of laboratory adapting a microorganism to an increasingly higher salinity environment is already described herein. Additionally, a process for knocking out genes (e.g., a Large-conductance mechanosensitive channel gene) in microorganism is also described herein. Other methods known in the art for knocking out genes in microorganisms (e.g., gene editing) can also be used. A halotolerant microorganism of the disclosure may be engineered to express one or more heterologous genes and/or over express one or more endogenous genes and/or have a reduced or eliminated expression of one or more endogenous gene such that the microorganism produces a desired product (e.g., a chemical, protein, biofuel, polymer etc.).

In a particular embodiment, methods or processes for producing an engineered halotolerant microorganism disclosed herein that expresses a desired protein or compound comprises the step of transforming an engineered halotolerant microorganism disclosed herein with a vector encoding a desired recombinant protein or encoding a desired chemical compound. In a further embodiment, the engineered halotolerant microorganism is cultured to produce the desired recombinant protein or the desired chemical compound. In a further embodiment, methods or processes for producing an engineered halotolerant microorganism disclosed herein that expresses a desired protein or compound further comprises the steps of: transferring the engineered halotolerant microorganism to a hypotonic solution to lyse the microorganism; and isolating the desired recombinant protein or the desired chemical compound released from the engineered halotolerant microorganism. Methods for protein and compound production are known in the art and can be used with the methods and processes disclosed herein.

The processes and methods of the disclosure are broadly applicable, and can be used to generate a variety of biomacromolecule products from a variety of different types of microorganisms. Accordingly, the processes and methods of the disclosure can be used to generate engineered microbial strains for large scale protein, biomolecule, and biopolymer (e.g., polyhydroxbutyrate (PHB)) production. The processes and methods of the disclosure can be used to generate engineered microbial strains for basic science research.

The disclosure demonstrate laboratory evolution and rational genome engineering to produce strains that are sensitive to osmotic downshock, and therefore may be used for simplified, osmolysis-based downstream recovery. The disclosure further demonstrates that by deleting the mscL gene in C. necator, also increased the cell lysis efficiency following osmotic downshock from 19 to 62%. While the putative function of this gene in C. necator was inferred by homology to be a large-conductance mechanosensitive channel, the disclosure provides the first experimental evidence of its function. The disclosure also demonstrates that the combined strategies in a single strain, C. necator ht030b ΔmscL, exhibited the highest cell lysis efficiency of over 90% when grown in LB medium and 99% when grown in M9 formate, demonstrating the efficacy of combining adaptive laboratory evolution with rational mechanosensitive channel knockouts to enhance cell lysis efficiency.

These strategies were expanded to other organisms. For example, the techniques were used to develop a strain of E. coli BL21, a common strain for protein expression, that is susceptible to osmolysis. Two gene knockouts (mscL and mscS) were required for significant (˜75%) protein release.

The disclosure demonstrates that the implementation of the strategies leads to significant cell lysis in both E. coli and C. necator, allowing intracellular bioproducts to be easily recovered from the strains, using red fluorescent protein as a model product. Either of the strategies described here for increasing osmolysis should be broadly applicable, as many (especially non-marine) bacteria contain the mscL gene, and adaptive laboratory evolution is suitable for most bacterial strains.

The following specific examples are intended to illustrate, but not limit, the disclosure.

### EXAMPLES

Microbial Media and Culturing Methods. All E. coli strains were grown at 37° C. and all C. necator strains were grown at 30° C. unless otherwise stated. Luria Broth (LB) was used as media for all E. coli cultures unless otherwise stated. Media were supplemented with kanamycin (50 μg/mL E. coli, 200 μg/mL C. necator) and/or carbenicillin (100 μg/mL) as appropriate. All liquid cultures were shaken at 200 RPM. All bacterial strains used in the study (see Table 1) were stored at −80° C. in 25% glycerol until needed. To reactivate the strains, cells were first streaked onto agar plates (with appropriate antibiotic as needed), incubated, and then a liquid LB culture was started from a single colony.

TABLE 1
Strains and Plasmids used in this study

| STRAIN/PLASMID | DESCRIPTION |
|---|---|
| Strains | |
| C. NECATOR H16 | Wild-type Cupriavidus necator strain |
| C. necator H16 Δmscl | C. necator strain deficient in gene encoding large- |
| | conductance mechanosensitive channel |
| C. NECATOR ht030b | C. necator strain with improved halotolerance following |
| | 250 rounds of adaptive laboratory evolution |
| C. NECATOR ht030b Δmscl | Adapted halotolerant C. necator strain deficient in gene |
| | encoding large-conductance mechanosensitive channel |
| E. coli WM3064 | DAP-auxotrophic E. coli donor strain used for |
| | conjugation of C. necator |
| E. coli BL21 | E. coli B strain deficient in Lon and OmpT proteases |
| | widely used in protein expression |
| E. coli BL21 Δmscl | E. coli BL21-derived strain deficient in gene encoding |
| | large-conductance mechanosensitive channel |
| E. coli BL21 Δmscl ΔmscS | E. coli BL21-derived strain deficient in genes encoding |
| | large-conductance mechanosensitive |
| | channel and small-conductance mechanosensitive |
| | channel |
| Plasmids | |
| pBADTrfp | araBAD promoter, T7-stem loop, Kan^{R}, mRFP1 |
| | expression gene |
| pMQ30k | pMQ-30 derivative with Kan^{R} marker; SacB sucrose |
| | sensitivity gene; integrating plasmid used for gene |
| | deletion |
| pMQ30k-Δmscl | pMQ30k plasmid derivative containing 500 nt upstream |
| | and 500 nt downstream of mscL gene |
| PSIJ8 | Temperature-sensitive plasmid expressing lambda Red |
| | recombinase and flippase recombinase |
| | genes; AmpR; for gene deletion in E. coli. Addgene |
| | #68122 |

Adaptive Laboratory Evolution. The halotolerance of Cupriavidus necator was improved by adaptive laboratory evolution (ALE) in 10-mL batch cultures. Wild-type C. necator H16 was grown in 50-mL tubes with 10 mL LB medium with NaCl starting at 15 g/L (final concentration). After 24 h of growth, cells were passaged into 10 mL fresh LB medium in 50-mL tubes at an initial optical density of A₆₀₀=0.001. At the end of each passage, the average growth rate was calculated from the initial and final culture densities, assuming constant exponential growth. When the average growth rate either plateaued or exceeded 0.3 h⁻¹, the salt concentration of the culture was increased by 0.25% (w/v) NaCl. This was repeated for 30 passages. Cells were plated every several passages on LB Agar plates to ensure the cultures were free of contamination. The final passage was plated on LB Agar with 3% NaCl (w/v, final concentration) and a single colony was selected for further experiments, with the strain named C. necator ht030b.

The growth rate of strain ht030b was compared to that of wild-type C. necator H16 at elevated salt concentrations. Overnight cultures of each respective strain were inoculated into four 1-mL volumes of LB supplemented with 3% NaCl (w/v, final concentration) in a 24-well plate at a cell density of A₆₀₀=0.01 and grown overnight at 30° C. Absorbance measurements (600 nm) were taken every hour.

The ht30b strain from the adaptive laboratory evolution, was streaked on an LB plate containing 3% (w/v) NaCl. Two colonies were grown overnight in liquid LB containing 3% (w/v) NaCl, and the genome was purified from each sample using a Monarch® Genomic Purification Kit (New England Biolabs). A single colony of wild-type H16 was grown, and the genome was likewise purified as a control. Extracted genomic DNA was used to prepare 150 bp paired-end Illumina sequencing libraries. These libraries were then sequenced using a NovaSeq 6000 S4 Sequencing System. Single nucleotide polymorphisms and other genomic variants were determined using requisite applications within the Geneious Prime software (version 2022.2.2, www.geneious.com). SNP's/variants were called from mapped reads originating from three different samples: one sample of the unevolved, wildtype C. necator H16 strain, which served as input to the adaptive laboratory evolution, and two different samples of the evolved strain exhibiting elevated halotolerance.

Paired reads were first trimmed to remove low quality bases, filter out and remove short reads (<10 bp), remove sequencing adapter content, and trim/remove low complexity regions using the ‘Trim using BBDuk’ application within the software. The ‘minimum quality’ setting was set to 30 and all other parameters were left at their default values. Reads were then mapped to a C. necator H16 reference genome composed of GenBank accession numbers CP039287, CP039288, and CP039289 (the sequences associate with the accession numbers are incorporated herein by reference). The application ‘Map to Reference’ was used for this process with the Sensitivity set to ‘Medium-Low Sensitivity-Fast’ and all other parameters set to their default values. The application ‘Find Variations/SNP's’ was then used to identify variations within the mapped assemblies with all parameters set to their default values. Identified variants were then manually filtered to remove those that were not represented with at least 27× coverage, variant frequencies<90% among mapped reads covering the candidate variant position, and those exhibiting a strand bias<25% or >75%. Tandem repeat variants>5 bp were also filtered out and not considered further.

Variants were first called from reads originating from the unevolved C. necator H16 sample in order to identify differences between this assembly and the reference genome. Variations found to be unique to either of the evolved populations and not present in the unevolved C. necator H16 sample assembly, and meeting aforementioned criteria, were considered. Also considered were variations that were found in the C. necator H16 unevolved samples but not found in the evolved populations.

Transformation of plasmids to C. necator. C. necator strains were transformed with plasmids in a two-step method in which the plasmids were first transformed to chemically competent E. coli WM3064 cells by heat shock, followed by conjugation of the plasmid from the WM3064 donor strain to C. necator. Chemically competent WM3064 cells were made as follows. WM3064 cells were cultured in LB to an optical density of A₆₀₀=0.4. Cells were chilled on ice for 20 min before being pelleted via centrifugation at 4,000 g for 5 min. Cells were resuspended in an ice cold 100 mM CaCl₂ solution and incubated on ice for one hour. Cells were then centrifuged at 4,000×g and resuspended in a 100 mM CaCl₂ solution with 10% glycerol at 50× the original cell concentration. Cells were stored in 50 μL aliquots at −80° C. until needed.

All C. necator strains listed in Table 1 were transformed with the red fluorescent protein expression plasmid pBADTrfp, following the protocol from Windhorst et al. (Biotechnol. Biofuels 12, 163 (2019)) pBADTrfp (Addgene plasmid #99382; n2t.net/addgene:99382; RRID:Addgene 99382). The transformation was performed via conjugation using E. coli WM3064 as a donor. pBADTrfp was transformed into chemically competent WM3064 cells using heat shock and positive clones were selected for on LB-Agar plates containing diaminopimelic acid (DAP, 0.3 μM) and kanamycin (50 μg/mL) following a 1-hour outgrowth in SOC medium. The C. necator strain and the transformed WM3064 strain were both grown overnight in LB and LB with DAP and kanamycin, respectively. The following day, the two cultures were inoculated into a fresh culture of the same media and were allowed to grow until the optical density of each culture reached A₆₀₀˜0.5. The WM3064 culture was washed twice with LB DAP, and 0.75 mL of each culture were mixed together. This mixture was pelleted by centrifugation at 8,000×g for 10 min, resuspended in 100 μL of LB-DAP, plated on a nitrocellulose membrane on an LB DAP plate and incubated at 30° C. for 18 hours. The filter was then resuspended in 2 mL of LB with kanamycin (200 μg/mL) and 50 μL was plated on an LB agar plate with kanamycin (200 μg/mL). The conjugation plate was incubated at 30° C. for 2 days. Proper transformation of the plasmid was confirmed via colony PCR.

Gene deletion in C. necator. Strains lacking the mscL gene were generated following a method relying on integrative plasmids and sucrose counterselection adapted from Windhorst et al. A gene fragment containing a 500 nucleotide region matching the region upstream of the mscL gene in C. necator H16 followed by a 500 nucleotide matching the region downstream was synthesized by IDT DNA Technologies. Overhang regions matching 20 nucleotide-long regions upstream and downstream of EcoRI and BamHI cut sites, respectively, were added by PCR, and the fragment was assembled with the linearized pMQ30k vector by Gibson Assembly, yielding the plasmid pMQ30k-ΔmscL. This plasmid was transformed into C. necator H16 and ht030b via conjugation with WM3064 as described above, and cells were selected on an LB agar plate with kanamycin (200 μg/mL). Kanamycin-resistant colonies were then grown overnight in liquid LB supplemented with kanamycin (200 μg/mL). This culture was then passaged in a 1000-fold dilution into LB without antibiotics and cultured for 24 hours. The cells were plated on an LB agar plate supplemented with 15% w/v sucrose for counterselection. Proper gene deletion was confirmed by colony PCR and Sanger Sequencing.

Gene deletion in E. coli BL21. The mscL gene was deleted from E. coli BL21 using a lambda Red recombination system. A gene cassette, containing a kanamycin resistance gene flanked by a flippase recognition target (FRT) site on each end, and with homology arms matching 120 bp upstream and downstream of the E. coli BL21 mscL gene on the 5′- and 3′-termini, was synthesized by Integrated DNA Technologies. Chemically competent BL21 cells (New England Biolabs) were transformed with pSIJ8, a temperature-sensitive plasmid that contains arabinose-inducible λ-Red recombinase genes and rhamnose-inducible flippase recombinase gene. pSIJ8 (Addgene plasmid #68122; n2t.net/addgene:68122; RRID:Addgene_68122). Strains containing the pSIJ8 plasmid were grown at 30° C. to maintain plasmid stability. BL21 pSIJ8 was grown in 15 mL of Terrific Broth (TB) medium (supplemented with carbenicillin) until reaching a cell density of A₆₀₀=0.35, followed by a 45-min induction with arabinose (2 mg/mL, final concentration). Cells were then made electrocompetent by four consecutive wash steps in chilled 10% (v/v) glycerol solution, which concentrated cells ˜100-fold. On ice, 5 μL (250 ng) of the synthetic DNA cassette were added to 50 μL of electrocompetent cells and cells were electroporated (1.8 kV, 1 mm gap, BTX Gemini X2). Cells were recovered with 950 μL TB for 3 h. Cells from the outgrowth were pelleted and resuspended in 200 μL TB, and cells were plated on LB Agar plates with kanamycin and carbenicillin and grown for 36 h. A single colony was selected and grown in LB with kanamycin and carbenicillin overnight. The following day, the culture was washed in LB, diluted to a cell density of A₆₀₀=0.1, and flippase expression was induced for 4 h with 50 mM L-rhamnose, which removed the integrated kanamycin gene from the BL21 genome. Serial dilutions were performed and cells were plated on LB Agar with carbenicillin and grown overnight. Correct gene deletions were verified by colony PCR. This strain was saved for further experiments. The mscS gene was then deleted from the BL21 ΔmscL strain using a similar DNA cassette with homology arms matching 120 bp upstream and downstream of the E. coli BL21 mscS gene, following the same protocol. The plasmid pSIJ8 was then cured from both BL21 ΔmscL ΔmscS and BL21 ΔmscL by growing them overnight in LB at 37° C. without antibiotics.

RFP-Based Cell Lysis Assay. A red fluorescent protein (RFP)-based lysis assay was developed to measure the osmolysis efficiency, or, the fraction of cells that are lysed upon resuspension in distilled water. The C. necator strain of interest was first transformed with the expression vector pBADTrfp, which contains an arabinose-inducible RFP gene, by conjugation. The C. necator strain carrying pBADTrfp was grown overnight in LB (final NaCl concentration 1.5% w/v for the non-halotolerant strain and 3.0% for the evolved strain). For experiments relying on heterotrophic growth, cells from the overnight culture were inoculated into LB (at appropriate salt concentration). In mid-exponential phase (A₆₀₀˜0.5), cells were induced with arabinose (final concentration 0.1% w/v) and RFP was expressed overnight at room temperature. For experiments relying on organoautotrophic growth, cells from the overnight culture (grown in LB) were inoculated into M9 minimal salts medium supplemented with 4 g/L sodium formate and either 6 g/L NaCl (for the non-halotolerant strain) or 16 g/L NaCl (for the halotolerant strain). Cells were grown overnight, pelleted, and resuspended in fresh medium containing arabinose (final concentration 0.1% w/v). RFP was expressed overnight at room temperature.

For both heterotrophic and organoautotrophic experiments, following overnight expression, the cells were washed once in their respective growth media and the cell concentration was adjusted to A₆₀₀=1.0. Cells were aliquoted in 1 mL volumes and centrifuged at 4,000×g for 10 minutes. Cells were resuspended in an aqueous solution containing various NaCl concentrations representing either an isotonic or hypotonic solution, and were shaken at 30° C. for 30 minutes. Two 150 μL samples were taken from the well-mixed cell solution and the fluorescence intensity was measured by a Spark Tecan microplate reader (Ex. 585 nm/Em. 620 nm). The rest of the cell solution was centrifuged at 4,000×g for 10 minutes and two 150 μL samples of the supernatant were taken and their fluorescent signal was measured as before in order to quantify the amount of RFP released to the extracellular space. The ratio of fluorescent signal in the supernatant and cell solutions was taken to be the fraction of cells lysed.

RFP-Based lysis assay for E. coli BL21 cells. E. coli strains BL21 ΔmscL and BL21 ΔmscL ΔmscS were made chemically competent following the same protocol as for WM3064, except for the omission of DAP. Chemically competent BL21 ΔmscL ΔmscS, BL21 ΔmscL, and wild-type BL21 cells were transformed with pBADTrfp via heat shock and selected for on LB Agar plates with kanamycin (50 μg/mL). Osmolysis experiments were performed following the same protocol as for C. necator, with minor adjustments. Overnight cultures were regrown at 37° C. in LB supplemented with the appropriate NaCl concentration. In mid-exponential phase (A600˜0.5), cells were induced with arabinose (final concentration 1 mg/mL) and RFP was expressed for 3 h at 30° C. The rest of the osmolysis assay follows the exact protocol as for C. necator. Samples from the total cell fraction, post-lysis supernatant, and cell pellet were saved for SDS-PAGE.

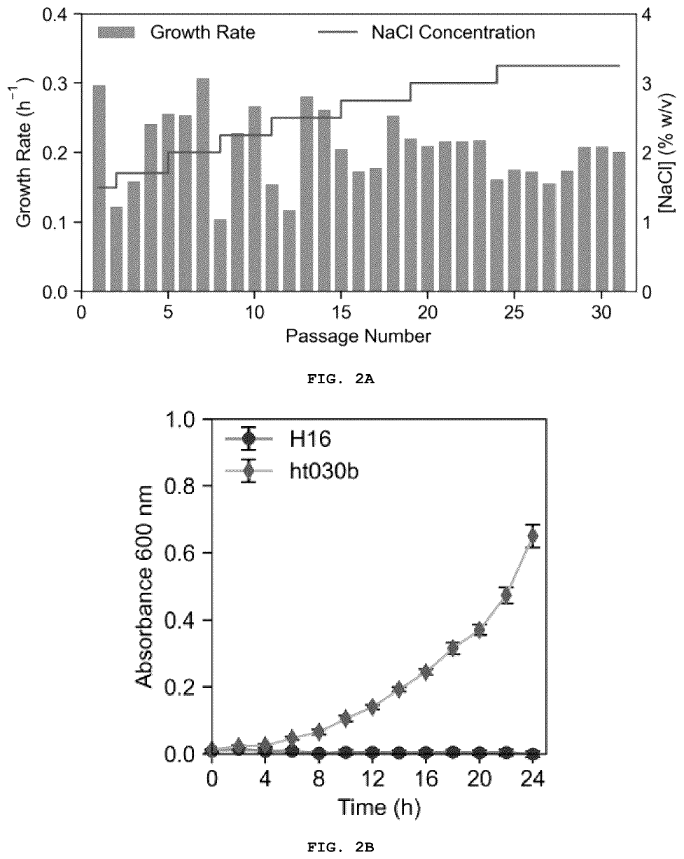

A halotolerant strain of C. necator evolved to grow in 3.25% w/v NaCl. Through 30 passages of ALE, accounting for roughly 250 generations of growth, the tolerance of C. necator in NaCl was improved from 1.5% to 3.25% (FIG. 2A). Microbial behavior during the ALE process was typical compared to studies performed previously. Sharp decreases in growth rate following addition of the stress (in this case NaCl) were regularly observed. The steepest increases in cell fitness were observed in early passages, with growth rates mostly plateauing in later passages. For example, the NaCl tolerance of C. necator improved by 1.25% (from 1.5% to 2.75%) in the first 15 passages, while only improving an additional 0.5% in the later fifteen passages. The osmolarity of 3.25% (w/v) NaCl, where the ALE began to plateau, is nearly identical to that of seawater. Given the existence of extremely halophilic proteobacteria such as Halomonas sp., it is plausible that the halotolerance of C. necator could improve with further rounds of ALE. However, these adaptations may occur significantly more slowly than the initial improvements observed here.

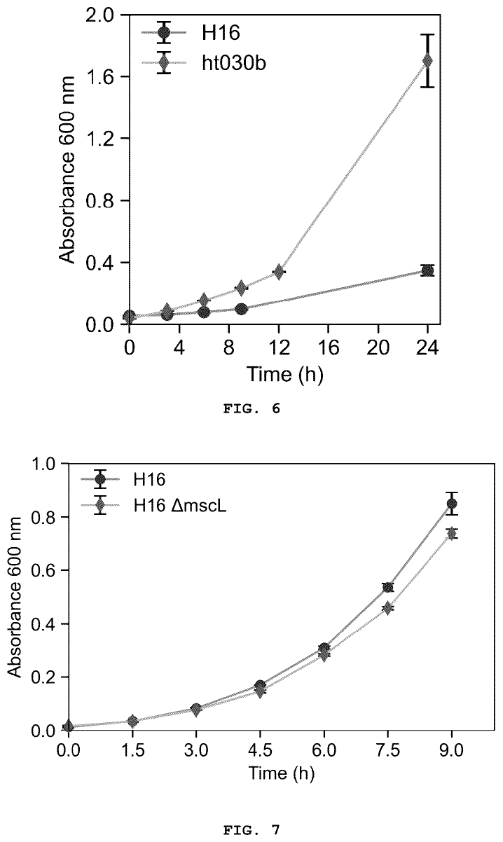

The growth of evolved strain ht030b, in comparison to the wild-type H16 strain, was tested in LB containing a (final) concentration of 3.25% NaCl (FIG. 2B). Under the conditions tested, the wild-type H16 did not display measurable growth over a 24-h period while strain ht030b grew with a growth rate of 0.16 h-1. It should be noted that when seeded with a high enough starting cell concentration, C. necator H16 displays some growth in LB with 3.25% NaCl, albeit at a significantly lower rate as compared to ht030b (FIG. 6). In any case, the strain generated through the ALE displays a clear phenotypic difference from the wild-type strain. This growth rate is lower than the growth rate of wild-type C. necator in LB containing the standard 0.5% NaCl (0.45 h-1).

Genomic analysis of ht030b. Genomic sequencing and the subsequent variant analysis identified five mutations (meeting the filtering criteria applied) that were acquired by strain ht030b throughout the course of the ALE (Table 2). Four mutations were present only in the ht030b genome, but not in either the reference H16 genome used for mapping or in the unevolved H16 genome sequenced. Each caused substitutions of a single amino acid in a different protein: a PAS domain-containing sensor histidine kinase NtrY, a peptidoglycan D,D-transpeptidase mrdA, an acetolactate synthase gene, and an IS66 family transposase gene. One SNP, causing a point substitution in a YgcG family protein, was surprisingly found only in the H16 genome, but not in the evolved strain ht030b or in the reference genome. This indicates that this mutation was present in the starting H16 strain but reverted to the original sequence as found in the reference genome throughout the course of the ALE. Unfortunately, the function of this gene family is unknown (although it is predicted to have transmembrane domains), limiting the understanding of the mechanism by which this could affect halotolerance.

TABLE 2
Mutations Detected in H16 and ht030b strains

| Chromosome | | | Mutation | | SNP |
|---|---|---|---|---|---|
| (NCBI | Genome | Gene/ | (CDS | | Present |
| Accession No.) | Coordinates | Encoded Protein | position) | Effect | In* |
| 1 | 3982709 | PAS domain- | G→A | Substitution | ht030b |
| (CP039287) | | containing sensor | (1606) | (R→C) | |
| | | histidine kinase | | | |
| 1 (CP039287) | 131230 | mrdA | C→T | Substitution | ht030b |
| | | Peptidoglycan | (1688) | (T→I) | |
| | | D,D- | | | |
| | | transpeptidase | | | |
| 1 (CP039287) | 791503 | YgcG family | C→A | Substitution | H16 |
| | | protein | (757) | (G→W) | |
| 2 (CP039288) | 829590 | Acetolactate | C→T | Substitution | ht030b |
| | | synthase | (1490) | (T→I) | |
| pHG-1 | 138984 | IS66 family | T→A | Substitution | ht030b |
| (CP039289) | | transposase | (206) | (E→V) | |

The mrdA gene encodes an enzyme in the penicillin-binding protein 2 (PBP2) family, and is involved in the synthesis in the peptidoglycan cell wall in bacteria, particularly in cell elongation. Penicillin-binding proteins have been shown to be important to cell survival under conditions of high salt stress, perhaps in response to the morphological changes (e.g., cell plasmolysis) that occur during osmotic upshifts. Recruitment of an mrdA homolog to the cell division site in Caulobacter crescentus in response to increases in salinity, suggests the regulation of this gene is involved in the osmotic stress-response mechanism. Therefore, it is conceivable that a change in either expression level or activity due to the detected mutation in the mrdA gene causes structural changes of the cell wall in C. necator ht030b, leading to enhanced survival in high-salt conditions. Interestingly, mutations of this type seem to be highly conserved in bacterial species. Strain ht030b displays a T563I mutation, substituting the polar amino acid threonine with a branched chain nonpolar amino acid isoleucine in that position. Notably, 63 out of the 72 diverse bacterial species represented in the mrdA protein family model in the TIGRFAM database (TIGR03423) contain a branched chain amino acid (I, L, or V) at that position, while only three contain a polar residue.

A thiamine pyrophosphate-binding protein with putative acetolactate synthase activity was found with a mutation in ht030b. Acetolactate synthase catalyzes the first step in the synthesis of branched-chain amino acids. A study of cells from diverse origins has shown a negative correlation between halotolerance and the intracellular concentration of branched chain amino acids (Sevin et al., PLoS ONE 11:e0148888, 2016). Amino acids and amino acid derivatives can be accumulated as compatible solutes as a mechanism of halotolerance. Therefore, changes to enzymes involved in the synthesis of amino acids could divert metabolic fluxes leading to the synthesis of these compatible solutes.

The histidine kinase gene (NtrY) mutated in the ALE is predicted to regulate nitrogen metabolism and the assimilation of nitrate. The NtrXY system and the related NtrBC system have been shown in other species to regulate the production and degradation of nitrogen-containing compounds such as arginine and ectoine, which again may affect osmotolerance due to the regulation of compatible solutes. In short, several of the unique mutations accumulated by strain ht030b through ALE could conceivably impart halotolerance.

Deletion of mscL gene from C. necator enhances cell lysis. A putative mscL gene was identified in the C. necator genome by a protein BLAST search of the homologous gene found in E. coli. The entire gene was deleted from C. necator H16 and successful ΔmscL mutants were screened by colony PCR and verified by Sanger Sequencing. Deletion of the mscL gene led to no obvious deleterious effects on the microbe, besides the desired sensitivity to osmotic downshock. Growth curves of both wild-type and mutant strains were obtained under identical conditions (FIG. 7). The growth rate of the mutant H16 ΔmscL (0.43±0.01 h-1) was not statistically different from the growth rate of wild-type H16 (0.45±0.01 h-1), indicating that the genome deletion did not impact overall cellular fitness.

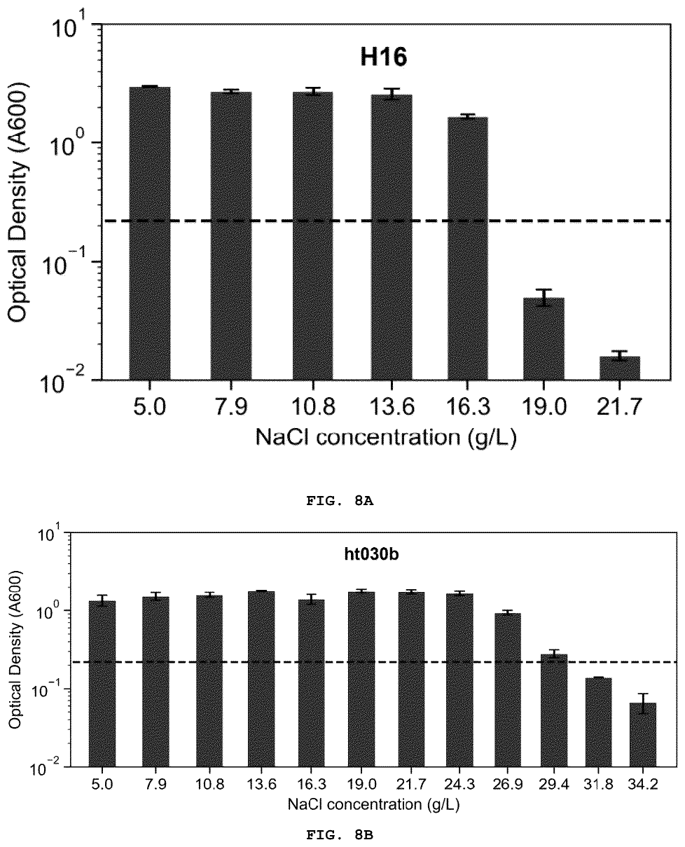

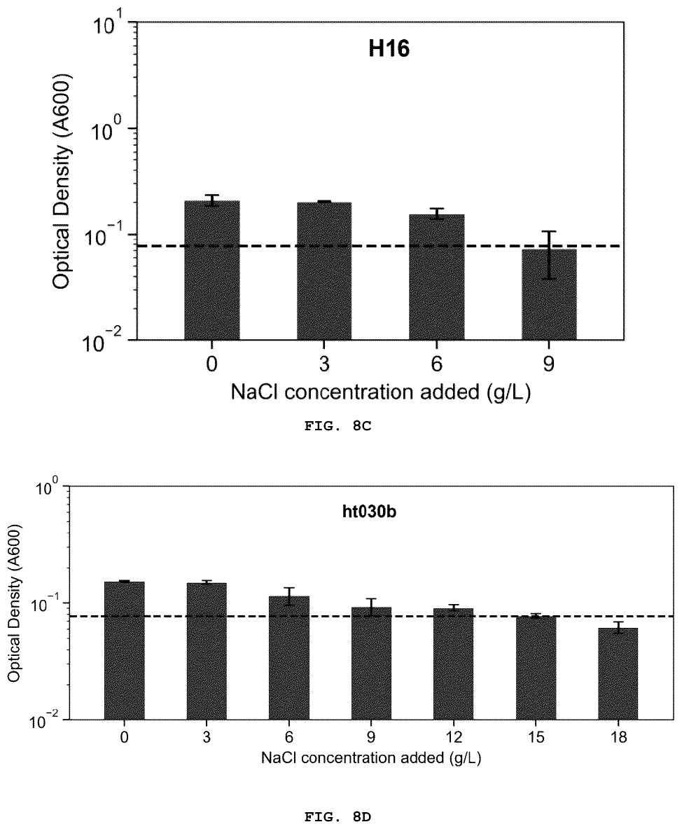

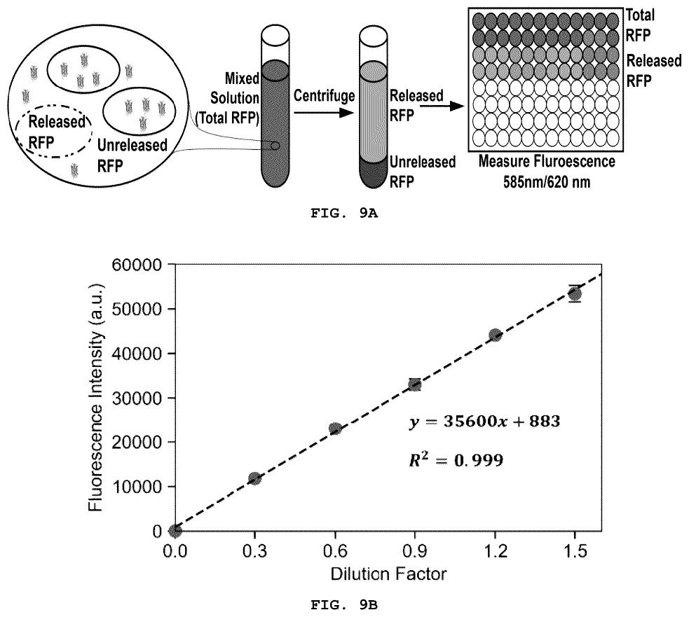

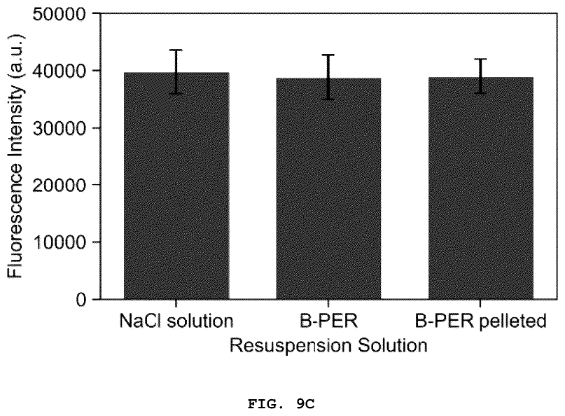

Both wild-type H16 and the mutant H16 ΔmscL were transformed with pBADT-rfp, containing the RFP gene under an inducible arabinose promoter, and the effect of the mscL gene deletion on the fraction of cells lysed upon osmotic downshock was tested. LB was supplemented with NaCl such that the final concentration of NaCl in the medium was 1.5% (w/v). This was the highest salt concentration at which the wild-type C. necator H16 strain still displayed measurable overnight growth (FIG. 8), maximizing the possible magnitude of osmotic downshock while still enabling functional cell growth. Red fluorescent protein (RFP) was expressed in both strains overnight, and washed cells were resuspended either in distilled water or an aqueous solution of 0.5%, 1%, or 1.5% (w/v) NaCl. After 30-min incubations, cells were pelleted and the ratio of red fluorescence intensity found in the supernatant to the fluorescence intensity of the whole solution was taken to be the osmolysis efficiency, or the fraction of cells lysed due to osmotic downshock.

For both wild-type and knockout strains, the highest osmolysis efficiency was observed when cells were resuspended in distilled water, as this caused the highest magnitude of osmotic downshock (0.51 OsM) and therefore the highest osmotic pressure (1.3 MPa). However, significantly greater cell lysis efficiencies were achieved with the mscL knockout strain (62%) compared to the wild-type (19%). This demonstrates that the native function of the putative mscL gene in C. necator is involved in the cell survival response following osmolarity changes, as it is in other bacteria. Following downshock, most of the cells in the wild-type C. necator sample remain intact, whereas a majority of cells are lysed when the gene is deleted. Deletion of this gene, therefore, is a simple strategy to increase the susceptibility of microbial hosts and aid in the recovery of intracellular biomolecules.

Similar levels of background cell lysis, which we define as cell lysis observed when resuspended in an isotonic solution, are observed in both the mscL+ and ΔmscL strains (<5%). Therefore, the mscL gene knockout does not make C. necator significantly more fragile under normal conditions. The increase in cell lysis only occurs upon osmotic downshock. This, along with the lack of change in the growth rate in H16 ΔmscL mentioned previously, suggests that the mscL gene is not critical to C. necator survival under normal conditions.

This experiment was repeated in defined medium using sodium formate as a sole carbon and energy source to evaluate osmolysis following autotrophic growth. Although there is interest in using C. necator for bioproduction under heterotrophic conditions, autotrophic production of biomolecules using molecules such as formic acid or hydrogen gas as energy sources enables electromicrobial production. C. necator was grown in M9 mineral medium with 4 g/L sodium formate as a carbon source with variable concentrations of added sodium chloride. The maximum amount of NaCl that could be added to M9 medium was determined to be 6 g/L (FIG. 8). The osmolarity of the medium with this much added salt (0.49 OsM) is close to the osmolarity of LB with 1.5% NaCl (0.51 OsM). Nearly identical trends were seen in experiments with LB and M9 formate, with both H16 and H16 ΔmscL experiencing greater osmolytic efficiencies as the magnitude of osmotic downshock increased. The mutant strain lysed significantly more than the wild-type strain (60% vs 18%) when resuspended in distilled water, similar to the LB experiment. While not surprising, as the mscL gene is not known to affect cellular metabolism and therefore the effect of its absence should be indifferent towards the carbon metabolism used, this does demonstrate that this strategy can be used to aid downstream recovery for both heterotrophic and autotrophic processes.

Osmolysis efficiency of C. necator ht030b and ht030b ΔmscL. The evolved halotolerant strain of C. necator (ht030b) was transformed with the plasmid pBADTrfp and osmolysis was tested following growth in LB supplemented with 3% NaCl (w/v, final concentration) following the same fluorescence-based lysis assay used in the previous section for C. necator H16 and H16 ΔmscL. Cells were resuspended in aqueous solutions ranging from 0% (distilled water) to 3% NaCl in 0.5% increments and lysis efficiency was measured as before (FIG. 4A). As expected, the maximum cell lysis (47%) was observed after resuspension in distilled water, corresponding to an osmotic pressure change of 1.03 OsM. This is more than double the lysis efficiency observed when resuspending wild-type C. necator H16 in distilled water (19%, FIG. 3A). Adapting bacteria for growth in higher salinities allows for higher levels of osmolysis. Therefore, both strategies described in FIG. 1, adapting the microbial host to greater halotolerance and deleting the large-conductance mechanosensitive channel gene, led to enhanced cell lysis in C. necator.

These two strategies were then combined in a single strain by deleting the mscL gene from the evolved ht030b strain. Successful gene deletion was confirmed by colony PCR and the resultant strain was transformed with the RFP-expressing plasmid. The experiment performed on ht030b was then performed on this new strain. As with the unevolved C. necator, deletion of the mscL gene significantly enhanced the fraction of cells lysed, with over 90% osmolysis efficiency observed when ht030b ΔmscL was resuspended in distilled water (FIG. 4). The combination of the two strategies described led to a greater osmolysis efficiency than either strategy independently. While the osmolysis efficiency of wild-type C. necator was only 19% at its maximum, nearly complete lysis of ht030b ΔmscL was achieved (compare FIGS. 3A and 4A). The combination of ALE to increase halotolerance and the gene deletion of mechanosensitive channels is clearly an effective method for engineering osmolytic susceptibility in a microbial host, which can greatly simplify downstream bioprocessing.

Interestingly, the magnitude of osmotic downshock, and therefore the magnitude of osmotic pressure, does not alone determine the cell lysis efficiency. For both mscL+ and ΔmscL strains, significantly greater cell lysis occurred upon moving cells from 1.5% NaCl(aq) to distilled water (19% for mscL+ and 62% for ΔmscL) than from 3.0% to 1.5% NaCl(aq) (8% for mscL+ and 13% for ΔmscL), despite equivalent osmotic pressure changes in each scenario. Similar observations can be made in the experiments with formate media. The salinity of the resuspension media also plays a role in determining the efficiency of cell lysis. It's possible that certain membrane proteins take on different conformations in deionized water than they do under normal salt concentrations. Therefore, the cells would experience greater cell lysis in distilled water despite the same osmotic pressure change.

While C. necator ht030b grew moderately well in 3% NaCl under heterotrophic conditions (in LB), it did not grow as well at equivalent salinities during organoautotrophic growth. When M9 formate was supplemented with various concentrations of NaCl, C. necator ht030b did not significantly grow when the added NaCl concentration exceeded 16 g/L (FIG. 8). The total osmolarity of this medium was 830 mOsm/L, which is equivalent in ionic strength to a roughly 2.4% (w/v) NaCl solution. Moderate halotolerance is usually effected by the accumulation of compatible solutes, including sugars and amino acids (and their derivatives), inside the cell to balance osmotic pressure. Rich media such as LB contain an abundance of amino acids, which can easily be imported by the cell and used as compatible solutes directly or converted to compatible solutes. Therefore, it is not particularly surprising to see slight differences in halotolerance in the two media tested

Strains ht030b and ht030b ΔmscL were grown in M9 formate supplemented with 16 g/L NaCl, and the RFP lysis assay was performed. Various solutions ranging from distilled water to 2.4% NaCl were tested to measure cell lysis in response to various magnitudes of osmotic downshock. As in all other experiments, the deletion of the mscL gene leads to significantly greater osmolysis efficiencies, reaching 98% of ht030b ΔmscL cells compared to 49% of ht030b cells. These strategies for engineering susceptibility to lysis by osmotic downshock are thus useful not only for heterotroph-based bioprocesses, but autotrophic processes as well.

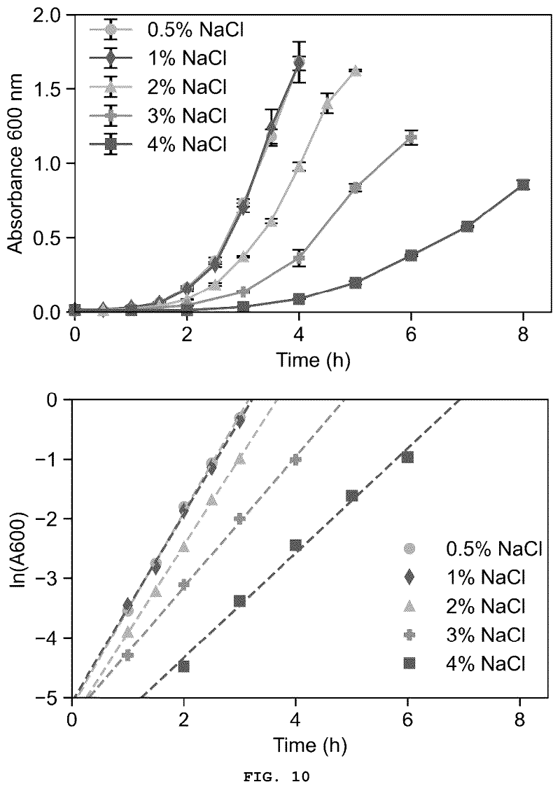

KO of mscL and mscS genes in E. coli BL21 enables significant protein release. Due to the success of the osmolysis strategy in C. necator, this technique was applied to other microbial hosts. E. coli BL21, a derivative of E. coli B strain deficient in Lon and OmpT proteases routinely used for production of recombinant proteins, was chosen as a second model system to evaluate this strategy. Because E. coli BL21 could already grow in elevated NaCl concentration, maintaining around half of its maximum growth rate even in 4% NaCl (FIG. 10), further adaptation of the strain was unnecessary. Therefore, only the effect of the mechanosensitive channel gene deletions was tested.

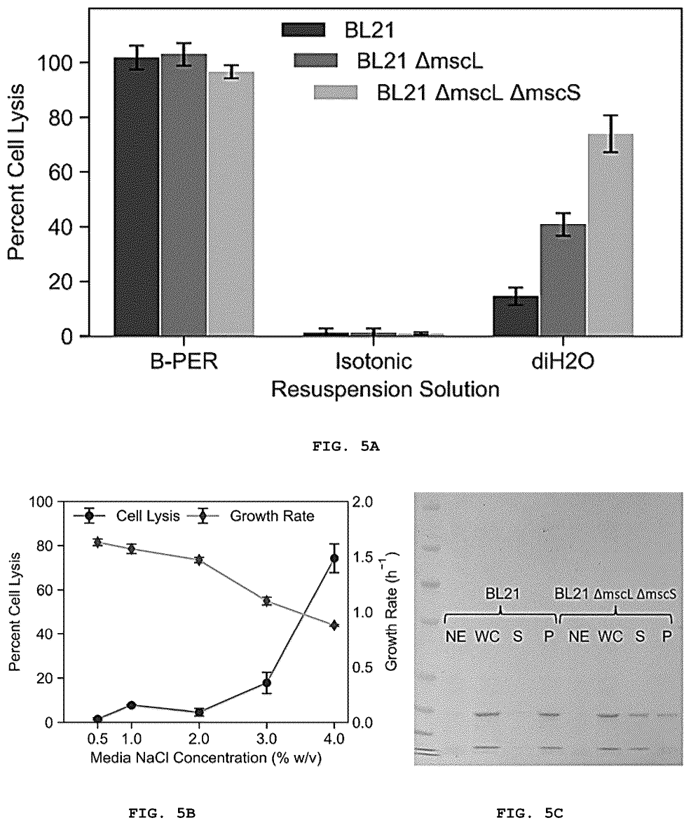

The mscL gene was successfully knocked out of E. coli BL21, as confirmed by colony PCR, and the resultant strain was transformed with the RFP-expressing plasmid pBADTrfp. The RFP-based osmolysis assay was then performed as it was for C. necator (with minor variations), comparing the engineered and wild-type strains. Cultures of these strains were grown in LB containing 4% (w/v) NaCl and cell lysis was tested following resuspension in distilled water, 4% NaCl isotonic aqueous solution, or B-PER™, a commercial bacterial lysis reagent (ThermoFisher Scientific) used as a positive control. As it did in C. necator, deleting the mscL gene from E. coli BL21 significantly increased the lysis efficiency in distilled water (41% v. 15%). Thus providing evidence that this strategy is broadly applicable. To improve the lysis efficiency, a second gene in the mechanosensitive channel family, the small-conductance mechanosensitive channel (mscS) gene, was also deleted from BL21. The double knockout demonstrated increased sensitivity to osmotic shock, reaching an average cell lysis efficiency of 75% (and efficiency as high as 81% in individual trials) following growth in LB with 4% NaCl (FIG. 5A).

Following the RFP-based assay, SDS-PAGE was performed on the whole-cell, supernatant, and cell pellet fractions post-osmolysis (FIG. 5C). Although more difficult to quantify exactly, the results of the gel are roughly consistent with the results of the RFP assay. Almost no protein is observed in the supernatant for the wild-type BL21 cells following resuspension in distilled water, while a significant fraction is for BL21 ΔmscL ΔmscS cells. It appears that a greater fraction of the total protein content is present in the supernatant fraction compared to the cell pellet fraction in the double knockout cells, consistent with the results of the RFP assay. Taken with RFP data, this demonstrates that the BL21 ΔmscL ΔmscS strain can be used to greatly simplify the recovery of expressed proteins, while maintaining a high protein recovery.

To test the effect of media salt concentration on osmolysis efficiency, this experiment was repeated in LB with NaCl concentrations of 0.5%, 1%, 2%, and 3% (w/v, FIG. 5B). It should be noted that 0.5% and 1% NaCl concentration are the two salt concentrations found in most LB formulations. Even in the double knockout strain, little more than background levels of cell lysis were observed for NaCl concentrations of 2% or less. Low (18%), but statistically significant (p<0.013), levels of cell lysis were observed for BL21 ΔmscL ΔmscS beginning at a media NaCl concentration of 3%. A sharp increase in the cell lysis efficiency occurred with an increase from 3 to 4% NaCl, suggesting the critical osmotic pressure required for cell lysis for most cells in the population falls within that range. As expected, the mutant E. coli strain experienced the greatest degree of cell lysis upon osmotic downshock following growth in the media with the highest osmolarity tested (4% NaCl). However, under these conditions, the growth rate of E. coli BL21 was significantly affected. A roughly linear decrease in specific growth rate of BL21 was observed with increasing NaCl concentrations. Increasing the salt concentration in the media from 0.5% to 4% w/v NaCl coincided with a growth rate decrease from 1.6 h-1 to 0.88 h-1.

In practical applications, a tradeoff would be encountered in which greater product recovery via osmolysis would come at the cost of a slower microbial growth rate. However, for many applications, a decreased growth rate may be worthwhile. The specific growth rate of 0.88 h-1 corresponds to a doubling time of roughly 45 min, which is still significantly faster than many microbial hosts. Routine lab-scale protein expression and purification protocols could still be performed in a single day, even at this diminished growth rate, as a seed culture inoculated to an optical density of A600=0.05 could reach mid-log phase (A600=0.5) in around 3 h. In continuous industrial-scale reactors, productivity is often limited by oxygenation rate rather than specific growth rate. Therefore, continuous bioreactor systems can likely operate at higher salt concentrations with a similar productivity despite the decrease in growth rate.

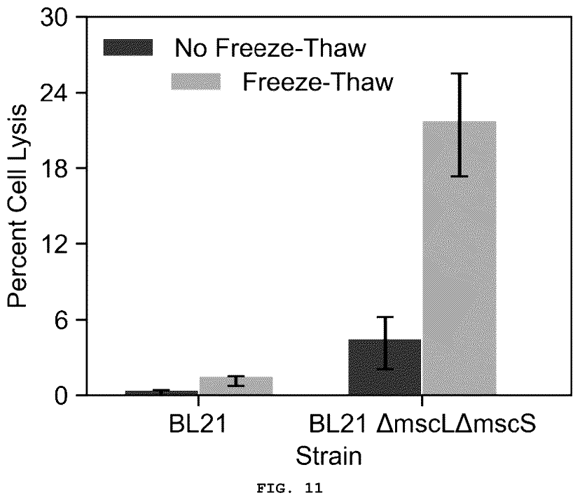

Although only a 75% cell lysis efficiency (on average) was attained in these experiments, this efficiency could be improved. Slightly higher NaCl concentrations in the media would likely be tolerated by BL21, and if the trend in FIG. 5B continues, one would expect near complete cell lysis could be achieved. Furthermore, this strain could also be used in conjunction with other cell lysis methods. For example, adding a single freeze-thaw step to the process increased the osmolysis efficiency of BL21 ΔmscL ΔmscS grown in LB with 2% NaCl from 4.5% to 22% (FIG. 11), while such an increase was not observed for BL21 without the gene deletions.

Certain embodiments of the invention have been described. It will be understood that various modifications may be made without departing from the spirit and scope of the invention. Other embodiments are within the scope of the following claims.

1. An engineered microorganism that is halotolerant or adapted to become halotolerant and comprises a knockout of a Large- and/or Small-conductance mechanosensitive channel gene.

2. The engineered microorganism of claim 1, wherein the microorganism can grow in greater than 1.5% w/v NaCl to about 4.0% w/v NaCl.

3. The engineered microorganism of claim 1, wherein the engineered microorganism can grow in greater than 2.0% w/v NaCl to about 4.0% w/v NaCl.

4. (canceled)

5. The engineered microorganism of claim 1, wherein the large-conductance mechanosensitive channel gene is mscL gene or a homolog thereof.

6. The engineered microorganism of claim 5, wherein the mscL gene has a sequence of SEQ ID NO:1.

7. The engineered microorganism of claim 5, wherein the mscL gene homolog is at least 80% identical to SEQ ID NO:1.

8. The engineered microorganism of claim 1, wherein the large-conductance mechanosensitive channel gene encodes a polypeptide that is at least 85% identical to SEQ ID NO:2.

9. The engineered microorganism of claim 1, wherein the microorganism is adapted to grow on a salt medium of 1.5% to about 3.25% (w/v) NaCl prior to knocking out the Large-conductance mechanosensitive channel gene.

10. The engineered microorganism of claim 1, wherein the engineered microorganism is further engineered to produce a non-natural chemical or bioproduct.

11. A method of producing a desired recombinant protein or chemical compound comprising transforming an engineered halotolerant microorganism of claim 1 with a vector encoding the desired recombinant protein or polypeptides that synthesize the desire chemical compound.

12. (canceled)

13. The method of claim 11, wherein the engineered microorganism is cultured to produce the desired recombinant protein or the desired chemical compound.

14. The method of claim 13, wherein the method further comprises:
transferring the engineered microorganism to a hypotonic solution to lyse the microorganism; and
isolating the desired recombinant protein or the desired chemical compound released from the engineered microorganism.

15. A method of generating a halotolerant microorganism from a non-halotolerant microorganism, the method comprising:
(a) passaging the non-halotolerant microorganisms on media that increases in salt concentration from by 0.25% (w/v) NaCl until reaching a final media concentration of about 1.5% to 3.0% (w/v) NaCl to obtain a laboratory evolved halotolerant strain;
(b) engineering the laboratory evolved halotolerant strain by genetically knocking out a gene(s) encoding a large and/or small conductance mechanosensitive channel protein(s) or a homolog thereof to obtain a halotolerant microorganism.

16. The method of claim 15, wherein the non-halotolerant microorganism is C. necator or E. coli.

17. (canceled)

18. The method of claim 15, wherein the large conductance mechanosensitive channel gene is mscL and wherein the small conductance mechanosensitive channel gene is mscS.

19. (canceled)

20. A halotolerant microorganism obtained by the method of claim 15.

21. The halotolerant microorganism of claim 20, wherein the microorganism has greater than 75-90% osmolytic efficiency upon osmotic downshock.

22. A halotolerant C. necator strain that grows on (i) 3% NaCl luria burtani broth (LB) or (ii) M9 formate with 16 g/L NaCl.

23. The halotolerant C. necator strain of claim 22, wherein the strain lacks expression of a large conductance mechanosensitive channel protein.

24. The engineered microorganism of claim 1, wherein the microorganism is a halotolerant E. coli strain comprising ΔmscL and ΔmscS.
